(19) United States
(12) Patent Application Publication
(10) Pub. No.: US 2016/0095637 A1
(43) Pub. Date: Apr. 7, 2016

(54) LOCKING MECHANISM FOR A CERVICAL FIXATION PLATE

(71) Applicant: Nexxt Spine, LLC, Noblesville, IN (US)

(72) Inventors: Andrew J. Elsbury, Fortville, IN (US); Eric J. Lintula, Parker, CO (US)

(21) Appl. No.: 14/874,443

(22) Filed: Oct. 4, 2015

Related U.S. Application Data
(60) Provisional application No. 62/059,953, filed on Oct. 5, 2014.

(51) Int. Cl. A61B 17/80; A61B 17/70

(52) U.S. Cl. CPC A61B 17/8047; A61B 17/8052; A61B 17/7059; A61B 17/7082

(57) ABSTRACT

A cervical plate includes a body defining one or more bone screw openings sized and configured to receive the shank of a bone screw therethrough with the head of the bone screw seated within the opening. A locking post extends from the body adjacent one or more of the bone screw openings with a locking element rotatably mounted thereon. The locking element includes a wing corresponding to each of the bone screw openings, with each wing sized to overlap a portion of the bone screw opening when the locking element is in a locking orientation and the head of a bone screw is seated within the bone screw opening. A swaged interface between the locking post and the locking element retain the locking element on the body while permitting the locking element to be rotated from a locking orientation to an orientation in which the bone screw openings are unimpeded to permit introduction of a bone screw.

### REFERENCE TO RELATED APPLICATION AND PRIORITY CLAIM

This application is a utility filing from and claims priority to U.S. Provisional Application No. 62/059,953, filed on Oct. 5, 2014, the entire disclosure of which is incorporated herein by reference.

### BACKGROUND

The present disclosure relates to orthopaedic fixation systems, and in particular to cervical fixation systems in which bone screws extend through openings in a plate configured to span two or more cervical vertebrae.

Fixation plates are often used to help stabilize the cervical spine, such as to assist an interbody implant(s) in the fusion of two or more adjacent cervical vertebrae. In the typical fixation procedure, the curved bottom surface of the cervical plate is placed on the anterior faces of the vertebral bodies and screws are driven through openings in the plate into the underlying bone.

One problem of cervical fixation systems is maintaining screw fixation in the bone, or more specifically preventing one or more bone screws from backing out of the vertebral body. Prior devices address this problem with complicated locking mechanisms or locking tools that can affect the cost, complexity, prominence and reliability of the fixation system. One further problem with prior locking mechanisms is that a special instrument is required to actuate the mechanism once the bone screw is seated within the plate and bone.

### SUMMARY

A cervical fixation plate comprises a body defining one or more bone screw openings sized and configured to receive the shank of a bone screw therethrough with the head of the bone screw seated within the opening. A locking post extends from the body adjacent one or more of the bone screw openings with a locking element rotatably mounted thereon. The locking element includes a wing corresponding to each of the bone screw openings, with each wing sized to overlap a portion of the bone screw opening when the locking element is in a locking orientation and the head of a bone screw is seated within the bone screw opening. A swaged interface between the locking post and the locking element retain the locking element on the body while permitting the locking element to be rotated from a locking orientation to an orientation in which the bone screw openings are unimpeded to permit introduction of a bone screw.

In another aspect, a tool is provided for rotating the locking element when the fixation plate is in situ. The tool includes a guide post that is received within a bore defined in the locking post to facilitate placement of the tool. The tool further includes an engagement body at the end of a tool shaft that is configured to engage the locking element. The engagement body defines opposite notches that can receive a corresponding wing of the locking element to permit rotation of the locking element by rotation of the tool.

### DESCRIPTION OF THE FIGURES

FIG. 1 is a perspective exploded view of a cervical plate according to the present disclosure.

FIG. 2 is a side cross-sectional view of the cervical plate shown in FIG. 1 shown in one step of assembly.

FIG. 3 is a side cross-sectional view of the cervical plate shown in FIG. 2 shown in a subsequent step of assembly.

FIG. 4 is a partial perspective view of the cervical plate shown in FIG. 1 with a locking tool.

FIG. 5 is a side cross-sectional view of the cervical plate shown in FIG. 4 with a portion of the locking tool engaged to the plate.

### DETAILED DESCRIPTION

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

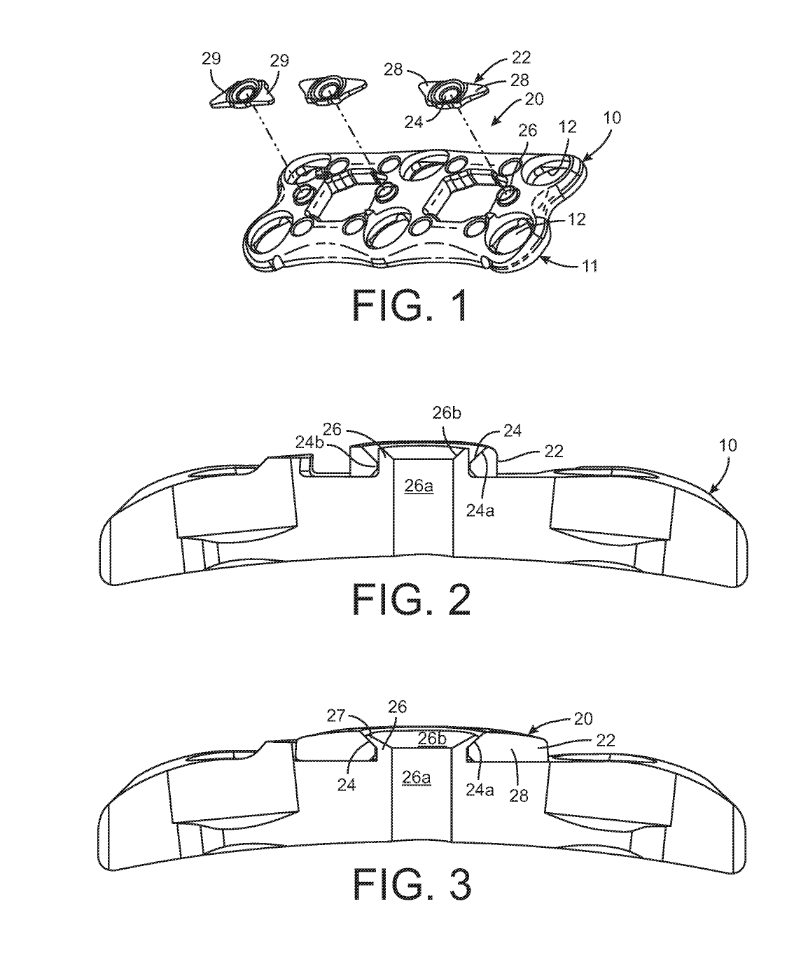

A cervical fixation plate 10 is provided with a locking mechanism 20, as depicted in FIGS. 1-3. The cervical plate can be configured in a conventional manner for engagement to a cervical vertebral body. In particular, the plate 10 is plate body 11 that can be curved in a known manner to provide a flush contact with the vertebral body being fixed. The plate body defines a plurality of bone screw openings 12 that are configured to receive the shank of a bone screw therethrough while the head of the bone screw is seated within the opening. The bone screw openings can be configured to seat the bone screws at a fixed angular orientation relative to the plate or at variable angular orientations relative to the plate. The locking mechanism 20 disclosed herein is adapted to retain any form of bone screw within the bone screw opening in the fixation plate. It is understood, however, that the locking mechanism 20 disclosed herein can be implemented on other types of orthopaedic fixation plates in which a bone screw is disposed within a recess defined in the plate.

In one aspect of the present disclosure, the plate is provided with a locking post 26 disposed between pairs of bone screw openings 12. In the illustrated embodiment, a locking post is disposed between all of the bone screw openings 12. However, it is contemplated that a locking post and associated locking mechanism 20 may be provided for a single bone screw opening or for less than all of the openings on a given fixation plate.

The locking post defines a bore 26a and a conical surface 26b. For manufacturing simplicity, the bore 26a can extend through the thickness of the body 11 of the plate 10, although it is understood that the bore 26a can be configured as a blind bore. A locking element 22 of the locking mechanism 20 includes a central opening 24 that includes a conical portion 24a and a cylindrical portion 24b that is sized for a close fit over the locking post 26, as shown in FIG. 2. The conical portion 24a is generally co-extensive with the conical surface 26b of the through bore 26a in the locking post. When the locking element 22 is mounted over the locking post 26, the conical surface 26b of the locking post can be swaged outward over the conical portion 24a of the central opening 24 in the locking element 22, as shown in FIG. 3. The locking element 22 is thus positively retained on the cervical plate 10 but is still permitted to pivot about the locking post 26. Moreover, the locking element and swaged post have a very low prominence above the surface of the plate 10.

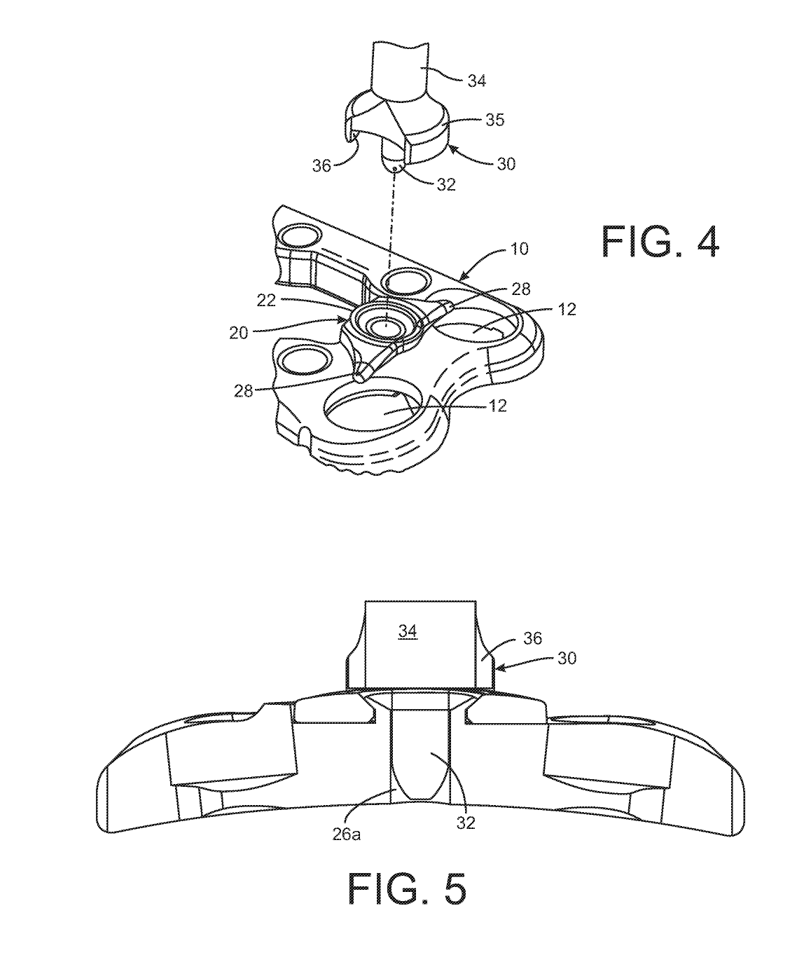

The locking element 22 includes opposite wings 28 that are arranged to overlap a portion of the bone screw openings 12 when screw heads are present within the openings. The wings 28 includes relief surfaces 29 that coincide with the outer circumference of the screw openings 12 when the locking element 22 is in a first orientation in which the wings extend across the width of the plate 10 with the relief surfaces 29 immediately adjacent a corresponding bone screw opening. In this orientation, the locking element does not encroach into the openings. The locking element can thus be rotated to the first orientation in which the wings 28 do not impede placement of a bone screw into each of the openings 12 (180° opposite to the orientation shown in FIG. 4). Once a bone screw or screws have been placed in corresponding openings 12 in the cervical plate 10, the associated locking element 22 can then be rotated to a second orientation shown in FIG. 4 in which the wings 28 overlap the openings and screw head disposed within the opening to prevent the screw from backing out. As shown in FIG. 4, the wings 28 are sized to only overlap a small sector or portion of the bone screw opening. More particularly, the wings 28 are sized so the wings are clear of a driving tool recess within the bone screw so that the surgeon can still access the driving tool recess even when the locking element 22 is in the second locking orientation.

The locking element 22 can be formed of a biocompatible material with sufficient rigidity to avoid bending under force from the bone screw should it attempt to back out of the vertebral bone. The locking element may be a metal, and particularly the same metal used to form the fixation plate to avoid any galvanic reaction between dissimilar metals. In one example, the locking element and plate can be formed of titanium or a medical grade stainless steel. Alternatively, the locking element may be formed of a biocompatible plastic or resin material that resists degrading over time.

The cervical plate 10 may be provided to the surgeon with the locking elements fastened to the plate—i.e., with the post 26 swaged onto the locking element 22. Preferably, the plate is provided with the locking elements in the first orientation described above in which the relief surfaces 29 are aligned with the bone screw openings 12. The surgeon can position the plate 10 on the vertebral body and introduce all of the bone screws through the openings 12 to engage the plate to the vertebral body. Once a pair of bone screws are disposed in an adjacent pair of openings, the associated locking element 22 may be rotated by a tool 30, as illustrated in FIGS. 4-5. The tool 30 includes a guide post 32 that is configured to extend into the passage 26a of the locking post 26 so that the tool can be easily and readily located in situ during a surgical procedure. The tool includes an engagement body 35 at the end of the tool shaft 34 that is configured to engage the locking element 22. The engagement body 35 defines opposite notches 36 that can receive a corresponding wing 28 of the locking element 22. The notches contact the wings upon rotation of the tool, to thereby rotate the locking element into the locked position shown in FIG. 4. The tool 30 includes a conventional handle that allows the surgeon to easily rotate the tool 180° to move the locking element between the first and second orientations as desired.

In the illustrated embodiment, the wings 28 are arranged 180° opposite each other to overlap bone screw openings that are themselves on directly opposite sides of the fixation plate 10. It can be appreciated that some fixation plates may include bone screw openings that are offset along the length of the fixation plate. In this instance, the wings 28 of the locking element 22 may be correspondingly offset from the 180° opposite position. It can be further appreciated that the locking element may be provided with three wings arranged at 120° intervals to overlap three similarly arranged bone screw openings.

In the illustrated embodiment, the locking post 26 projects from the surface of the fixation plate 10. Alternatively, the locking post may be situated within a recess with the locking element 26 seated within the recess. This configuration reduces the profile of the locking mechanism; however, the recessed locking mechanism will also reduce the height of the bone screw head that can be accepted within the bone screw opening of the plate.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

What is claimed is:

1. A cervical plate comprising:
a body defining one or more bone screw openings sized and configured to receive the shank of a bone screw therethrough with the head of the bone screw seated within the opening;
a locking post extending from said body adjacent one or more of said bone screw openings;
a locking element rotatably mounted on the locking post, the locking element including a wing corresponding to each of the adjacent one or more of said bone screw openings, said wing sized to overlap a portion of the bone screw opening when the locking element is in a locking orientation with the head of a bone screw seated within the bone screw opening; and
a swaged interface between said locking post and said locking element to retain said locking element on said body.

2. The cervical plate of claim 1, wherein:
said locking element defines an opening therethrough, said opening including a cylindrical portion sized for a close running fit with a portion of said locking post and a conical portion; and
said locking post includes a conical surface swaged against said conical portion of said locking element.

3. The cervical plate of claim 1, wherein said wing defines a relief surface configured to provide clearance around said bone screw opening when the in an orientation rotated 180° from said locking orientation.

4. The cervical plate of claim 1, wherein:
said body defines a pair of bone screw openings adjacent each other;
said plate includes a locking post disposed between said pair of bone screw openings; and
said locking element rotatably mounted on said locking post is configured with a wing simultaneously overlapping a corresponding one of said pair of bone screw openings when the locking element is in the locking orientation.

5. The cervical plate of claim 4, wherein locking element includes a pair of wings that are 180° opposite each other.

6. The cervical plate of claim 1, wherein said locking post defines a bore for receiving a post of a tool adapted to rotate the locking element relative to the plate.
